(19) United States
(12) Patent Application Publication
(10) Pub. No.: US 2009/0042280 A1
(43) Pub. Date: Feb. 12, 2009

(54) FLUIDIC CARTRIDGES FOR ELECTROCHEMICAL DETECTION OF DNA

(75) Inventors: Xing Yang, San Diego, CA (US); Robert Erik Holmlin, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: GENEOHM SCIENCES, INC.,

(21) Appl. No.: 11/721,533

(22) PCT Filed: Dec. 6, 2005
(86) PCT No.: PCT/US2005/044271

§ 371 Date: Nov. 1, 2007

Related U.S. Application Data
(60) Provisional application No. 60/635,552, filed on Dec. 13, 2004.

(51) Int. Cl. C12M 1/34; C12Q 1/00

(52) U.S. Cl. 435/287.2; 204/403.03

(57) ABSTRACT

A flow cell cartridge for the detection of differences in nucleic acid sequences is disclosed. The flow cell cartridge has an electrode array and two openings, in which one opening is for the entry and exit of sample, and the other opening is for the control of the entry and exit of sample through the exertion of negative and positive pressure. The entire flow cell cartridge can be moved from sample to sample to allow different samples to be drawn into the cartridge into contact with an electrochemical electrode array, thus allowing reactions to occur in the chamber itself.

### FIELD OF THE INVENTION

This invention relates to flow cells in electrochemical detection arrays for the detection of biological materials. In particular, the invention relates to an electrochemical detection array for detecting mutations in genetic material and is configured for the rapid and efficient exchange of the genetic material samples across the array.

### BACKGROUND OF THE INVENTION

Various techniques for detecting mutations in genetic material are known in the art. For example, techniques for detecting colorectal cancer are disclosed in U.S. Pat. No. 5,741,650 (Lapidus, et al.); U.S. Pat. No. 5,834,181 (Shuber); U.S. Pat. No. 5,849,483 (Shuber); U.S. Pat. No. 5,952,178 (Lapidus et al.); U.S. Pat. No. 6,268,136 (Shuber et al.); U.S. Pat. No. 6,303,304 (Shuber et al.); U.S. Pat. No. 6,428,964 (Shuber).

One method of detecting nucleic acid hybridization is through electrochemical techniques. Electrochemical quantitation is described in A. B. Steel et al., Electrochemical Quantitation of DNA Immobilized on Gold, Anal. Chem. 70:4670-77 (1998). In this publication, Steel et al. describe the use of cobalt (III) trisbipyridyl and ruthenium (III) hexamine as species which interact with surface-immobilized DNA.

Electrochemical assays for disease diagnostics involve multiple steps of washing, incubating, temperature cycling, as well as adding and removing ingredients from the assay chamber. Typically, the turnover or exchange of one reagent for another reagent in such a system is through the use of a flow device. One possible design of a flow device is shown in FIG. 1. In FIG. 1, the flow device, 10, has a flow chamber 40 in which an assay is performed. The flow chamber 40 is connected to various reagents or samples through different fluidic channels 20 and 30. The samples move though the flow chamber 40 via positive pressure exerted via the fluidic channels 30 and 20. The samples leave the flow chamber 40 via liquid permeable fluidic channels 70 and 80. The source of the positive pressure is usually obtained by using pumps, valves, and other fluidic devices, usually exerting some positive pressure on the reagent to be added to the flow chamber, which in turn forces out the reagents currently in the flow chamber. This type of device 10 is relatively complex and can involve fluidic devices such as pumps and valves, as well as other devices. Because of this complexity, this type of system is believed to be expensive and unreliable. Additionally, the flow chamber and the array are stationary, that is, the samples or reagents must be brought to the chamber, usually resulting in the elimination of some amount of the previous sample volume. Other forms of flow chambers use centrifugal force (e.g., Careside, Inc., Culver City, Calif.) or vacuum (e.g., Accumetrics, San Diego, Calif.) to move the reagents instead of positive pressure.

### SUMMARY OF THE INVENTION

In one aspect, a flow cell cartridge (FCC) is provided. The flow cell cartridge comprises a housing forming a chamber. The housing has a first opening adapted for the flow of a liquid and a second opening adapted for the flow of a gas. The openings open to the inside of the chamber and movement of a gas out of the second opening is adapted to move a gas or a liquid into said first opening. The flow cell cartridge further comprises an electrode array positioned inside the chamber such that a probe on the electrode array is exposed to the inside of the chamber. The electrode array has an electrically conductive connection to a point external to the chamber and at least a portion of the electrode array has probe nucleic acid bound to it. In one embodiment, the probe nucleic acid comprises peptide nucleic acid segments. In one embodiment, the chamber is shaped in two dimensions in a diamond shape. In one embodiment, the housing is configured to accept a pipette tip over a portion of the housing section creating the first opening. In one embodiment, the chamber has a first opening that is gas permeable and liquid permeable, and the chamber has a second opening that is gas permeable but not significantly liquid permeable. In one embodiment, the electrically conductive connection to the exterior of the chamber is configured to contact spring loaded pins. In one embodiment, the electrically conductive connections to the electrode array comprise pre-deformed metal bumps. In one embodiment, flow cell cartridge further comprises a Peltier device, a temperature sensor, and the temperature sensor is positioned within the chamber. In one embodiment, the flow cell cartridge further comprises a resistive heater and a temperature sensor.

In another aspect, a flow cell cartridge (FCC) is provided. The flow cell cartridge comprises a housing, the housing comprises 1) a front half and 2) a back half; the front half when joined with the back half forms 3) a chamber. The housing has a first opening that is both liquid and gas permeable and a second opening that is gas permeable but is not significantly liquid permeable. The FCC further comprises at least one working electrode and a reference electrode, wherein the electrodes are in the chamber. The FCC further comprises an electrical connection located on an external surface of the housing, wherein said electrical connection is in electrical contact with one or more of the electrodes. In one embodiment, the flow cell cartridge further comprises a Peltier device thermally coupled to the chamber. In another embodiment, the FCC further comprises a temperature sensor thermally coupled to said chamber. In another embodiment, the FCC further comprises a nucleic acid probe attached to the working electrode. In another embodiment, the FCC further comprises a peptide nucleic acid segment attached to the working electrode. In another embodiment, the FCC further comprises a counter ion. In another embodiment, the counter ion is a ruthenium ion. In another embodiment, the back half of the housing comprises a substrate for an electrode. In another embodiment, the second opening is connected to a vacuum source. In another embodiment, the first opening is connected to a pipette tip.

In another aspect, a method of applying a sample to an electrode array is provided. The method comprises contacting a first opening of a flow cell cartridge (FCC) to a sample to be tested. The FCC comprises a housing that comprises a chamber, said chamber having a first opening and a second opening, and an electrode array contained within the chamber. The method further comprising applying a negative pressure to the second opening for a period of time to move the sample through the first opening and into the chamber, thus promoting contact between the sample and the electrode array. In one embodiment, the method further comprises incubating a sample or reagent solution in the chamber for a time sufficient for polynucleotide indicative of the presence of analyte in a sample to bind to a probe nucleic acid attached to said electrode, and then expelling said sample or reagent solution from the chamber by exerting a positive pressure through said second opening. In another embodiment, the method further comprises applying a negative pressure to the second opening to bring into the FCC a set of reagents for performing a rolling circle amplification and performing a rolling circle amplification inside of the chamber. In another embodiment, the method further comprises waiting for a period of time sufficient to allow a target nucleotide segment in the chamber that is indicative of the presence of analyte in the sample to bind to a probe nucleic acid attached to the electrode, and determining an electrical signal at the electrode that is indicative of the presence of the target polynucleotide segment.

In another aspect, a method of performing an assay for the detection of a nucleic acid segment is provided. The method comprises contacting a first opening of a flow cell cartridge (FCC) to a sample to be tested. The FCC comprises a housing, the housing comprises a chamber, the chamber has a first opening, a second opening, an electrode array contained within the chamber, and a magnetic bead within the housing. The magnetic bead binds to a target nucleic acid segment. The method further comprising applying a negative pressure to the second opening for a period of time sufficient to bring the sample through the first opening and into the housing, allowing the sample in the housing to bind to the beads in the housing, expelling the unbound sample from the FCC into a waste well by applying a positive pressure to the second opening while maintaining the magnetic beads in the FCC, moving the FCC to a well containing a PCR solution, placing the first opening in contact with the PCR solution, applying a negative pressure to the second opening for a period of time to sufficient to bring the PCR solution into the housing, controlling the temperature of the PCR solution in the FCC to perform a PCR reaction, eliminating the PCR solution from the FCC, moving the FCC to a well containing a counter ion, placing the first opening in contact with the counter ion, applying a negative pressure to the second opening for a period of time sufficient to bring a counter ion through the first opening and into the chamber of the sample through the first opening, and detecting the electrical potential at the electrode, thus allowing the detection of a nucleic acid segment.

In another aspect, a method for performing an assay is provided. The method comprises providing a housing having a first opening, a second opening, a chamber interposed between the first and second opening, wherein the chamber includes at least one binding moiety for indicating a positive assay. Further providing at least first, second, and third wells containing liquids or reagents useful in performing an assay. Further positioning the first opening in liquid contact with the first well and moving liquid from the first well through the first opening and into the chamber by removing gas from the second opening. Further positioning the first opening in liquid contact with the second well and moving liquid from the second well through the first opening and into the chamber by removing gas from the second opening. Further positioning the first opening in liquid contact with the third well, moving liquid from the third well through the first opening and into the chamber by removing gas from the second opening, and ascertaining whether the binding moiety has participated in binding indicative of a positive assay. In one embodiment, the positioning steps comprise moving the wells relative to the first opening of the housing. In another embodiment, the positioning steps comprise moving the first opening of the housing relative to the wells. In another embodiment, the positioning steps comprise incrementally rotating either the housing or the wells. In another embodiment, the wells are covered with a frangible material that is pierced by movement of the first opening into fluid contact with the well. In another embodiment, one of the liquids moved into the chamber comprises a biological sample. In another embodiment, the biological sample contains, as an analyte, a nucleic acid. In another embodiment, the chamber contains an electrode array. At least one of the electrodes has a probe nucleotide that comprises the binding moiety. In another embodiment, a positive assay is indicated through generation of an electrochemical signal indicative of binding between the probe nucleotide and a nucleotide tag. In another embodiment, the method further comprises generating a nucleotide tag in response to the presence of analyte nucleic acid in a sample. In one embodiment, the nucleotide tag is generated through rolling circle amplification. In one embodiment, the nucleotide tag is generated through PCR. In one embodiment, the nucleotide tag is a target polynucleotide in a sample.

In one aspect, a fluidic cartridge system for the simple and rapid application of a sample to an electrode array is provided. The system comprises a flow cell cartridge (FCC) that comprises a housing with a first and a second opening and a chamber connected to both said first and second openings. The chamber is located within the housing. The chamber comprises an electrode array that comprises nucleic acid segments. The system further comprises a separate reagent cartridge that comprises wells that contain a sample that is accessible to the first opening of the flow cell cartridge. In one embodiment, the reagent cartridge is a rotational array. In another embodiment, the reagent wells are covered by a thin layer of protective material.

In another aspect, a flow cell cartridge (FCC) is provided. The flow cell cartridge comprises a housing. The housing supports an electrode array. The electrode array is positioned on an exterior surface of the housing so that at least one electrode of the electrode array can directly contact a sample. The electrode array has an electrically conductive connection, and at least a portion of the electrode array has probe nucleic acid bound thereto. In one embodiment, the housing further comprises a protrusion from an external surface of the housing, wherein said protrusion is located next to the electrode array. In one embodiment, the protrusion is configured to support a small volume of solution in contact with at least one electrode.

In another aspect, a fluidic cartridge system for the simple and rapid application of a sample to an electrode array is provided. The system comprises a flow cell cartridge that comprises a housing. The housing supports an electrode array that is positioned on an exterior surface of the housing so that at least one electrode of the electrode array can directly contact a sample. The electrode array has an electrically conductive connection, and at least a portion of the electrode array has probe nucleic acid bound thereto. The system further comprises a separate reagent cartridge. The reagent cartridge comprises wells that contain a sample that is accessible to an electrode in the electrode array on the flow cell cartridge. In one embodiment, the reagent cartridge further comprises a device that agitates a fluid in the reagent cartridge. In one embodiment, the device is a sonicator. In one embodiment, the device is a gas line attached to a well in the reagent cartridge so as to send bubbles through the fluid in the reagent cartridge.

In one aspect, a method of detecting a nucleic acid segment in a sample is provided. The method comprises contacting an electrode of a flow cell cartridge with a sample. The flow cell cartridge comprises a housing. The housing supports an electrode array. The electrode array is positioned on an exterior surface of the housing so that at least one electrode of the electrode array can directly contact the sample. The electrode array has an electrically conductive connection and at least a portion of the electrode array has probe nucleic acid bound thereto. The sample is contained in a first well of a reagent cartridge. The contacting of the electrode with the sample results in at least some of the sample being associated with the flow cell cartridge upon removal of the flow cell cartridge from the sample. The method further comprises contacting the electrode of the flow cell cartridge with a counter ion. The counter ion is stored in a second well of the reagent cartridge. The method further comprising measuring an electrical potential of the electrode.

In another aspect, a method of performing an assay for the detection of a nucleic acid segment from a cell is provided. The method comprises collecting cells in a pipette tip, lysing the cells in the presence of magnetic beads, wherein the magnetic beads comprise a nucleic acid segment binding agent that binds to a nucleic acid segment. The method further comprising adding a wash buffer to the pipette tip, removing the wash buffer, while maintaining said beads in the pipette tip through the use of a magnetic field, and adding amplification reagents and enzyme reagents to said pipette tip. The method further comprising removing the amplification and enzyme reagents from the pipette tip, while maintaining said beads in the pipette tip through the use of a magnetic field. The method further comprising adding a buffer to remove the nucleic acid segment from the beads and removing the nucleic acid segment from the pipette tip while maintaining the beads in the pipette tip through the use of a magnetic field. The method further comprising contacting a first opening of a flow cell cartridge (FCC) to a nucleic acid segment. The FCC comprising a housing, the housing comprising a chamber, the chamber having a first opening, a second opening, and an electrode array. The method further comprising applying a negative pressure to the second opening for a period of time sufficient to bring the nucleic acid through the first opening and into the housing, moving the flow cell cartridge to a well containing a counter ion, placing the first opening in contact with the counter ion, applying a negative pressure to the second opening for a period of time sufficient to bring a counter ion through the first opening and into the chamber of the sample through the first opening, and detecting an electrical potential at the electrode, thus allowing the detection of a nucleic acid segment from a cell.

### BRIEF DESCRIPTION OF THE DRAWINGS

FIG. 1 is a depiction of one device that can be used to flow various samples into and out of a chamber.

FIG. 2 is a depiction of one embodiment of a flow cell cartridge.

FIG. 3A is a depiction of an alternative embodiment of part of a flow cell cartridge.

FIG. 3B is a depiction of the embodiment of FIG. 3A along the line A-A′.

FIG. 4A is a depiction of one embodiment of a flow cell cartridge.

FIG. 4B is a depiction of an alternative embodiment of a flow cell cartridge.

FIG. 5 is a depiction of a method of using one of the disclosed embodiments.

### DETAILED DESCRIPTION OF THE INVENTION

Embodiments of the present invention include a flow cell cartridge (FCC) in which the detection of mutations in genetic material can be performed through the use of an array. The flow cell cartridge allows one to move various samples and reagents across the electrochemical detection array by moving the FCC itself to various samples and reagents, rather than by moving various samples and reagents to the flow device and then across the detection array within the flow device. This can simplify the flow device greatly and can also result in a flow device that requires a lesser volume of sample or reagent to contact the sample over the detection array. Additionally, as the FCC can be much simpler than other flow devices, the FCC can be more reliable and cheaper to produce.

Embodiments further include a flow cell cartridge comprising an analyte-responsive area, which can preferably be an electrochemical electrode array. In one embodiment, the entire electrode array is positioned in a chamber in the FCC so that a sample taken into the FCC can readily contact the electrode array. In one embodiment, the FCC is configured so that it can be moved from a first sample to a second sample; thus, allowing sample selection to be achieved through the movement of the FCC. In another embodiment, the flow cell cartridge comprises a chamber created between a substrate, on which the electrode array or electrodes are placed, and a second half of a housing. This embodiment allows for a greater degree of minimalization of the sample volume required as only the nucleic acid detecting sections of the electrodes are within the housing.

Embodiments further include reagent cartridges (RC), which can contain reagents that can be collected or removed from the flow cell cartridge. Embodiments include, for example, linear and rotational reagent cartridge arrays.

Embodiments further include the combination of the FCC and the RC. Embodiments further include the use of the FCC and the RC in combination to rapidly, efficiently, inexpensively, and simply move samples or substances over an electrode array. As used herein, an electrode array comprises at least two, preferably at least three or four, and more preferably at least 5, 8, 10, 15, 20 or more electrodes. At least some of the electrodes are analyte-responsive, meaning that a signal is measurable at the electrode when an analyte is present in the assay. Various techniques for creating such a signal are imown in the art, and in preferred embodiments, the present invention is not limited to any particular signal generation technology; rather, the present invention provides a structure and method that can be used with a wide range of assay technologies.

Embodiments further include methods of using the FCC, either alone or in combination with a RC, to detect the presence of target nucleic acid sequences in a sample. In one embodiment, the FCC is used on a relatively isolated form of target nucleic acid, such as a sample containing various RNA segments. In another embodiment, the FCC is used on a relatively impure form of target nucleic acid, such as a biological sample from a patient.

### Flow Cell Cartridge (FCC)

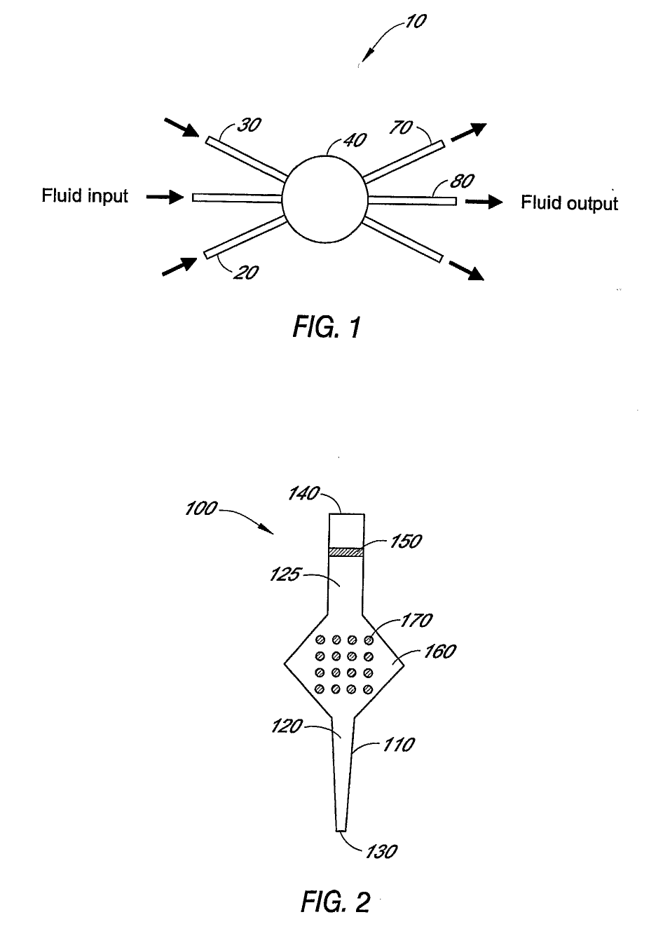

One embodiment of a flow cell cartridge 100 is shown in FIG. 2. In some embodiments, the FCC 100 comprises an electrochemical electrode array 170 within a housing 110. The housing 110 can be readily manipulated from a first location in a first sample or reagent to a second location in a second sample or reagent. In some embodiments, the flow cell cartridge has a housing that allows for reagents and other materials to be pulled into and expelled from the chamber without the need for additional sample to be added to the chamber. For example, the FCC can have one opening 130, through which sample can be added and removed, and a second opening 140, through which pressure at the first opening can be adjusted to allow the control of the movement of sample through the first opening. In one embodiment, the shape of the housing 110 creating the first opening 130 is adapted to fit on one end of a pipette tip and the shape of the housing forming the second opening 140 is adapted to fit on the end of a pipette or other pressure control device so that the device can be used to control the movement of sample across the electrode array 170 of the FCC. In one method of the present invention, the entire housing can be moved to each reagent or sample to allow for the materials to be added to the chamber 160, rather than in arrangements such as in FIG. 1, where each of the reagents is individually brought first to the flow device and then into the chamber 40.

The flow cell cartridge 100, has a housing 110 with a first opening 130 and a second opening 140. Between the first and second opening is a chamber 160 which can contain an electrode array 170. Additionally, there can be an optional passageway 120 from the first opening 130 to the chamber 160. There can also be such a passageway 125 from the chamber 160 to the second opening 140. Optionally, there is a filter 150 positioned between the chamber 160 and the second opening 140. The filter can be gas permeable and can advantageously be substantially or significantly liquid impermeable.

The housing 110 can be made of any material as long as the appropriate liquid and gas tight seals are achievable so that the housing functions properly to maintain the sample effectively close to the electrode array 170. In some embodiments, the housing is made of plastic. In some embodiments, the housing is treated with chemicals to prevent sample from sticking to the walls of the housing 110 or to prevent contamination or degradation of the sample. For example, amounts of RNase and DNase inhibitors can be useful to preserve sample quality. In some embodiments, the housing is part of a disposable or single use FCC, and thus, appropriately disposable materials can be used. For example, materials which are highly inert or highly sterile, but difficult to clean or reuse, can be used. The material can also be particularly free of contaminants, for example, RNase or DNase free materials. In one embodiment, the materials are highly resistant to changes in temperature, thus allowing the temperature to be adjusted within the housing itself. In one embodiment the housing material allows the rapid transfer of heat, thus permitting the sample temperature to be controlled through an external source. Alternatively, the housing material can be an insulator, reducing the impact of the external environment on the internal sample. The housing material can be a hydrophobic material, to allow the minimization of amount of sample retained on the housing surface. In one embodiment, the housing is made of a transparent material so that the sample inside the FCC can be visualized, for example, high purity polypropylene. The interior of the housing can also be coated with a material to achieve any of the described properties.

The actual shape of the housing can vary as long as it allows for the function of the housing 110 and the aspects of the FCC relevant to a particular selected end use. For example, while FIG. 2 demonstrates a housing that has a conical passageway 120, continuous with a diamond shaped chamber 160, continuous with a columnar second passageway 125, any shape which achieves the result of being able to control the flow of a liquid over an electrode array can be used. For example, the entire housing could be in the shape of a column, cone, diamond, tube, or other suitable shape, as long as a force applied to the second opening can control flow at the first opening sufficiently to get the sample to the electrode array 170. In one embodiment, the housing has additional characteristics such as magnetic or electromagnetic properties so that magnetic or metal beads can be associated with the interior of the housing. In some embodiments, a magnet is externally associated with the FCC such that magnetic beads can be associated within the FCC. In some embodiments, the magnet is a permanent magnet; in other embodiments the magnet is an electromagnet. Additionally, the three-dimensional shape of the interior of the housing can be varied as well. While the exterior of the housing for passageway 120 can be shaped to receive pipette tips, and thus, for example, can be conical, the shape of the inside of the passageway 120 and 125 and of the chamber 160 do not need to be conical or cylindrical. In one embodiment, the interior of the housing is minimized so as to require as little sample volume as possible to allow the sample to contact the electrodes or other analyte-responsive area. For example, the FCC 100 in FIG. 2 can effectively be flat or rectangular in the dimension in the plane of the page for the shape depicted. Such an effectively flat FCC could minimize the volume needed to cover the electrode array and thus allow for a more efficient system. Alternatively, the thickness of the walls of the housing can be adjusted to further minimize the amount of sample required. In some embodiments, the volume of liquid drawn into and expelled from the FCC is no more than about 10 ml; in other embodiments, it is no more than about 7 ml, 5 ml, 3 ml, 1000 μl, 500 μl, 100 μl, or even down to 10 μl.

As shown in FIG. 2, within the housing 110 is a first opening 130 and a second opening 140. The shape and location of each opening is not critical, as long as it allows sample to be drawn into the housing 110 to make contact with the electrode array 170 (or other analyte-responsive structure), preferably uniform contact. In one embodiment, the size of the first opening is large enough to allow the rapid addition and removal of sample from the FCC, while being small enough so as to minimize any sample lost from the FCC while the FCC is being moved from one sample to another. Additionally, a smaller opening could require a smaller sample size in order to move the sample across the array. Of course, the application of a negative pressure to the second opening 140 while a sample is in the FCC will also aid in preventing sample loss. As an example, suitable cross-sectional areas for the size of the first opening can be from about 5, 3, or 1 mm² down to about 100 or 50 microns in diameter or smaller. In one embodiment, there are multiple first openings 130, allowing multiple samples to be taken into the FCC simultaneously. In this embodiment, the multiple openings 130 can each be connected to a separate chamber 160 and electrode array 170, or alternatively, each of the multiple openings 130 can be linked to a single chamber 160 and electrode array 170. One possible advantage of having multiple openings 130 includes increased speed and a reduced need for vacuum sources.

The second opening 140 can be of any size or shape as long as it allows a sufficient amount of vacuum to effectively work the FCC 100. The relative positions of the first 130 and second openings 140 can also be varied. In one embodiment, the openings are positioned so that the sample intake opening 130 will allow the flow of sample to the electrode array, while the second opening 140, will avoid any passage of the sample. In one particular set of embodiments, the cross sectional areas of the second opening 140 is from about 20% to about 400% of the cross-sectional area of the first opening 130, and more preferably from about 50% to about 250% of the area of the first opening 130. However, the openings are preferably positioned such that application of reduced pressure or vacuum to the second opening draws sample into chamber 160 of the FCC that contains the electrode array 170. In another embodiment, the sample drawn into the electrode array can then pass through the second opening 140 to exit the FCC 100. Such a device can allow a simpler application of force to the FCC.

In some embodiments, the FCC 100 can also comprise a filter 150 which will substantially prevent the movement of a sample from the chamber 160 through the second opening 140 and into the source of the pressure, whether it be positive or negative pressure. Any type of filter can be used, as long as it allows sufficient gas to pass through the filter so as to allow control of the movement of sample to the electrode array. Filters that are similar to those used in pipette tips can be used. The filter can be relatively impermeable to the sample or solvents applied to the electrode so that it can stop or reduce the flow of the sample to the second opening 140. As will be appreciated by one of skill in the art, the method of using the FCC can be such that a filter is not required. For example, the amount of negative pressure applied to the sample can always be kept beneath an amount required to bring the sample through the second opening 140. In some embodiments, the filter 150 allows a sufficient amount of gas to flow through the filter so that a change in pressure at the first opening 130 is achieved that is capable of controlling the flow of liquid into and out of the chamber 160. In some embodiments, the filter 150 prevents any liquid from reaching the side of the filter that is opposite of the chamber 160. In some embodiments, the filter 150 prevents an amount of liquid that would be problematic to the methods performed through the use of the FCC 100 from passing through the filter. Thus, for example, in methods that allow a substantial amount of sample or solution carry over from one step to the next, the filter can be appropriately permeable to liquids. Alternatively, the filter 150 can be supplemented with or replaced by an optical sensor that ceases aspiration of liquid into the FCC when liquid is detected at the sensor, thus preventing aspiration of liquid into equipment connected to the second opening 140.

The FCC 100 can also have a chamber 160 inside the FCC and located between the first and second openings 130, 140. The chamber in the embodiment in FIG. 2 is a diamond shaped chamber. This particular embodiment of a chamber is one that assists in the removal of sample from the area with the electrode array. This shape can also help to minimize the volume needed to saturate the electrode array, and helps to mix the various samples together. However, the shape of the chamber, 160 is not critical and can vary depending upon the particular application of the FCC. In some embodiments, the chamber 160 is diamond-shaped to facilitate reagent draining. The chamber can be in other conformations as well. For example, the chamber can be spherical, columnar, rectangular, or cone shaped. In some embodiments, the chamber 160 is the same size and shape of the rest of the flow cell, in other words it has no distinct structural characteristics apart from the fact that the electrode array 170 or other analyte detection structure is positioned within it. The chamber 160 and the electrode array 170 can be positioned anywhere within the FCC 100. One advantage of placing the electrode array 170 at or close to the first opening of the FCC 130 is that a minimal amount of sample or reagent is required to provide contact of the sample with the electrode array 170. As discussed above, the chamber dimensions can vary depending upon the application; however, in one embodiment, the dimensions of the chamber 160 are 3 mm wide, 20 mm long and 1 mm thick. Thus, the total volume of the chamber is 60 microliters. In other embodiments, the total volume of the entire interior volume of the FCC 100 is less than about 100 microliters, 80 microliters, 60 microliters, 40 microliters, 20 microliters, 10 microliters, or even less.

The chamber 160 is characterized by the location of the electrode array 170. Thus, even in a FCC 100 that is more or less uniformly cylindrical from the first opening 130 to the second opening 140, a “chamber” will exist in the volume of the FCC occupied by the electrode array 170. Additionally, in one embodiment, the chamber 160 can be located anywhere within the housing 110 as long as the administration of pressure (either positive or negative) to the second opening 140 results in the ability to control sample movement to and from the first opening 130 and the electrode array 170. Thus, in one embodiment, the electrode array 170 is positioned generally in the middle of the FCC 100. In another embodiment, the electrode array 170 is positioned generally at or adjacent to the first opening 130. In embodiments of FCCs that do not require positive or negative pressure, for example, embodiments that work through capillary action or simple hydrophilicity or wicking action, there need be no recognizable structure to be identified as a chamber 160. Furthermore, in those embodiments, there need be no recognizable first 130 and second openings 140 or housing 110 that encloses the electrode array 170.

In one such embodiment, the electrode array 170 is positioned on an external surface of the housing 110. In this embodiment, a movement of gas is not needed to bring a sample into contact with the electrode array 170, as the array can be contacted with a sample by simply dipping the exposed array into the sample. The electrode array 170 need not be completely exposed on all sides, as structures to help break seals or to help retain a liquid sample close to the electrode array 170 can also be included. For example, a spike placed next to the electrode array or a wall or surface extending above the electrode array, but not enclosing the electrode array can be useful for breaking seals and retaining a volume of sample next to the electrode array, especially when the FCC 100 is moved from one sample to the next. These embodiments can retain sample volume through capillary action. The exchange of solution across the surface of an electrode can be achieved through minimizing the size of the sample collected, movement of the sample solution, such as through sonication, bubbling or stirring, or movement of the FCC 100 itself. In some embodiments, the electrode array 170 is completely exposed on the external surface of the housing 110, and the housing is used as a means for moving the electrode array from one sample to the next sample. In some embodiments, the electrode array 170 is simply placed on the bottom of a moveable arm, thus allowing the electrode array to be moved from one well of a reagent cartridge to another with great speed and requiring very little volume of sample to cover the electrode array.

Another embodiment that does not require the application of positive or negative pressure is one in which the FCC 100 is dipped into containers of reagents, wash solutions, etc.; those liquids flow into the chamber; the liquids are then retained in the FCC 100 for a desired length of time by simply closing the second opening 140, and then are allowed to drain out by simply opening the second opening 140. It will be appreciated that as long as the second opening is closed, liquid inside the chamber will remain there, but as soon as the second opening 140 is opened or the chamber is otherwise vented, liquid will drain out of the FCC 100 under gravity pressure. Likewise, a large amount of liquid is prevented from flowing up into the chamber so long as the second opening 140 is closed, even if the FCC 100 is immersed in a liquid.

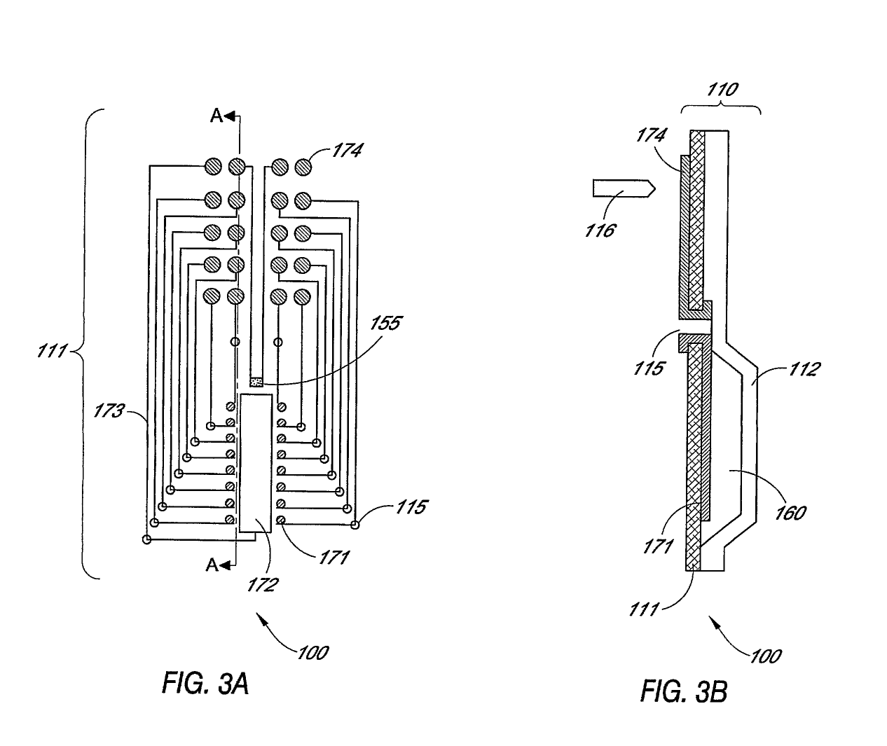

One embodiment of an electrode array is shown in FIG. 3A. An electrode array can have practically any number of electrodes. For example, the electrode array can have from 1 to 100 working electrodes. The number of electrodes is only limited by the ability to connect the electrodes to the housing. In each array or array system, there can advantageously be at least one reference electrode 172 and at least one detection electrode or carbon working electrode 171, although there can be more than one. In some embodiments, the electrode array 170 can be synthesized on a substrate or back surface 111. In such embodiments, the electrodes are on one side of the substrate, also known as a back surface 111, and pads 174 for connecting the electrodes to an electrical device are on the opposite side of the substrate. The electrical connection between the pads 174 and the electrodes 171 and 172 can be achieved through electrical conductors 173. The electrical conductors can cross from one side of the substrate 111 to an opposite side of the substrate via through-holes 115. The through holes 115 can be sealed so as to minimize sample loss if sample passes over the through holes to reach the electrodes 171 and 172. In one embodiment, the electrical connection itself seals the through-holes 115. In another embodiment, the through-holes 115 are positioned outside of the chamber 160 or the interior of the FCC 100 so that the presence of the hole does not alter the functionality of the FCC; an example of such a placement is shown in FIG. 3B. In one embodiment, the pads 174 and the electrodes 172 and 171 are on the same side of the substrate 111, thus, there is no need for through-holes 115. It will be apparent that conventional printed circuit board and semiconductor fabrication techniques can be utilized to form the electrical conductors, the pads, electrodes, and the like, if desired. Certain non-limiting embodiments of the electrode array 170 and a more complete description of the electrodes themselves and how they function are described in copending U.S. Pat. Pub. No. 20040086892, entitled “UNIVERSAL TAG ASSAY,” filed Apr. 24, 2003; U.S. Pat. Pub. No. 20040086894 entitled “ELECTROCHEMICAL METHOD TO MEASURE DNA ATTACHMENT TO AN ELECTRODE SURFACE IN THE PRESENCE OF MOLECULAR OXYGEN,” filed May 2, 2003.

In one preferred embodiment, the assay detects nucleic acid hybridization using the general technique of Steele et al. (1998, Anal. Chem. 70:4670-4677).

In some embodiments, a plurality of nucleic acid probes, or segments, that are complementary to a sequence of interest or a reporter sequence or tag are attached as part of an electrode. Preferably, these probe strands are immobilized on a surface such as an electrode, and are used in contact with a liquid medium. The area of the electrode that is associated with the nucleic acid probes (or any probing compound in general) is known as the probe part of the electrode. Preferably, the surface is a gold or carbon electrode that is coated with a protein layer such as avidin to facilitate the attachment of the nucleic acid probe strands to the electrode. The protein layer can be porous, such that it allows ions to pass from the liquid medium to the electrode and vice versa. Alternatively, probe strands can be attached directly to the surface, for example by using a thiol or other linkage to covalently bind nucleic acid to a gold or other electrode. Particular examples of such nucleic acids are discussed in more detail below.

In some embodiments of the FCC 100, the electrode array 170 (for electrochemical readout) can be bonded into a chamber 160 by attaching the electrode array to an interior wall of the housing, for example, as shown in FIG. 2. The electrode array 170 can be in electrical communication with other devices for observing the voltage at the electrodes via various means. For example, wires or other conductors (including printed or etched conductors) can be used to deliver an electrical signal from the electrode array to a device outside of the FCC. The FCC itself, and the electrical connection from the electrode array to the exterior of the housing 110, can be in electrical communication with other devices in any number of ways. For example, with reference to FIG. 3B, electrical connections to the electrode array 170 can be achieved by contacting spring-loaded pins 116, elastic conductive silicone bumps, or pre-deformed metal bumps to contact pads 174 on the exterior surface of the housing, as well as other means known to one of skill in the art. In some embodiments, the housing 110 is actually made up of a front half 112 and a chip substrate or back half 111. In this embodiment, the contacts or pads for the electrical connections 174 on the back of the chip 111 are exposed and the chip substrate (which can be a printed circuit board, for example) actually forms part of the housing itself (see FIG. 3A and FIG. 3B and the discussion below, for an example). In such an embodiment, the pads for the electrical connections 174 are on the exterior of the housing 110, as are the spring-loaded pins 116.

The external surface of the passageway 120, or the housing 110 in general, can also serve to allow additional devices to be attached to the end of the FCC 100, in particular to the end of the first opening 130. There are several possible devices that could be attached. In some embodiments, the housing 110 forming the passageway 120 is configured so as to allow a pipette tip to fit on the end, thus allowing a means to exchange tips. Thus, an angular housing, with a tapered first opening 130, as shown in FIG. 2, can be a desirable means to attach a pipette tip to the FCC. In addition to taper-based attachments, ridges, detents, threads, interlocks, and a variety of frictional attachments and the like can also be used to retain a pipette tip. Alternatively, a means for withdrawing a sample from a tissue can be added to the end of the passageway, such as a needle for withdrawing blood. Alternatively, the housing can be threaded so that a device can be screwed onto the tip. Alternatively, the tip can be magnetic or metal if the device to be added is metal or magnetic. There are many ways by which another device can be connected; the only requirement is that the connection create a sufficiently liquid tight seal so that sample is not lost, and a sufficiently air tight seal so that pressure applied to the second opening 140 can influence liquid movement within the FCC 100. It is noted that the exterior shape of the surface of the housing 110 that makes up the passageway 120 need not be defined by the interior surface of the passageway 120.

In some embodiments, the flow cell is connected to a syringe pump on the second end 140 and a pipette tip on the first end 130. The device can then be operated by using a syringe pump; no valves and other fluidic devices would be needed. This significantly reduces the complexity and cost of the system and increases its reliability. However, as will be appreciated by one of skill in the art, either of these could be altered. For example, instead of a syringe, a vacuum pump could be used or any source of a vacuum. Additionally, a means for exerting positive pressure could also be employed at the second end 140; for example, a pressurized canister of gas. One advantage of a syringe pump is that it can serve as both a source of vacuum and positive pressure for the FCC 100. In some embodiments, the device can be used with pipettes. By replacing the syringe pump with a pipette, an assay can be run on this cartridge as one would run solution into a pipette tip when working a pipette. In some embodiments, this is combined with commercially available 8-well strips as the reagent cartridge; this will provide a low-cost system for low volume tests. Additionally, such an embodiment will still allow both positive and negative pressure to be controlled from a single device. In some embodiments, the source of vacuum and the FCC 100 are a single piece of equipment.

Incubating the reagent at a certain temperature is a common step in assays. To achieve this, the FCC 100 can include a Peltier device or other heating and/or cooling structure. In some embodiments, the Peltier device is actually attached to the electrode array, on the chip substrate 111, for example. Additionally, a resistive heater and a thermistor can be fabricated on the chip surface. The devices can be positioned directly on or next to the electrode array. Alternatively, the devices can be positioned elsewhere in or on the surface of the housing 110. Additionally, a temperature sensor 155, can also be positioned within or on the FCC 100. In some embodiments, the temperature sensor 155 is positioned within the chamber 160, near the electrodes 172 and 171. In some embodiments, the temperatures of the electrodes themselves are used to determine the temperature of the solution in the chamber 160.

An alternative embodiment of a flow cell cartridge is depicted in FIG. 3A and FIG. 3B. In this embodiment, the housing of the flow cell cartridge comprises both a front half 112 and a back half 111. FIG. 3A depicts a view of a back half of a housing, or substrate 111. The front half of the housing 112 is removed to show other details of a FCC 100. FIG. 3B depicts both a back half of a housing 111 and a front half of a housing 112, which when combined, results in a complete housing 110, which forms a chamber 160 in which a sample can contact electrodes 171 and 172. In this embodiment, the housing 110 is in two parts, a front half 112 and a back half 111. This split can involve just the chamber 160, the area with the electrode array, or a larger part of the FCC, for example, the entire FCC housing 110 can be split in two parts. By halve or half, it is meant that there are two parts, not that the two parts must be equal in size. In some embodiments, the splitting of the housing 110 into two parts allows for greater ease of manufacture as placement of an electrode array 170 into a chamber 160 in this embodiment is relatively simple, as it only requires covering the surface of the electrode substrate 111 to create an effectively liquid and airtight seal. The splitting of the housing 110 can also facilitate minimizing the size of the chamber 160 and the entire interior volume of the FCC as the interior volume need be no larger than the dimensions of a sufficient electrode surface area and a sufficient height to allow sample flow and sample detection across the electrodes 170. Splitting the housing 110 can also allow for less material to be required in the creation of the housing, as the front of the housing 112 need only form a seal with the substrate 111, which also selves as the back half of the housing 111. The two halves can be assembled through the use of any number of appropriate manners; for example, a mechanical connection, RF welding, heat, or adhesives can be used.

### Reagent Cartridge

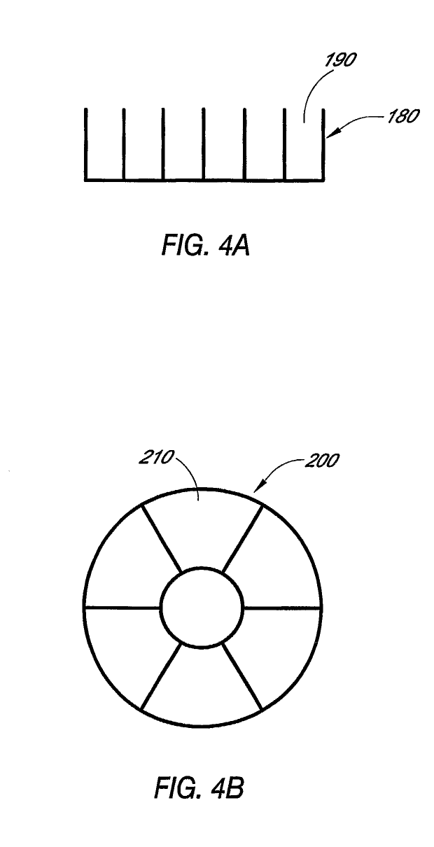

FIGS. 4A and 4B display two embodiments of a Reagent Cartridge. The reagent cartridge 180 is an array of compartments or wells 190. Various reagents, such as wash solutions, buffer, enzyme, substrate, or sample receptacles can be contained in the compartments 190. FIG. 4A displays a cutaway side view of a linear array cartridge 180, having at least six wells 190. FIG. 4B shows a top-down view of an alternative embodiment which encompasses a rotational array 200 having six or seven wells 210. Of course, any number and size of wells can be employed, and they need not be all the same shape or size. For example, 1-100 wells, or more, could be used.

In some embodiments, some or all of the compartments of the cartridge can be sealed with pierceable aluminum foil or other sealing tapes. These sealed reagent cartridges allow for the ready transportation and long term storage of reagents. In one embodiment, this allows for kits for nucleic acid detection via electrochemical detection to be created. Thus, all of the reagents required for sample collection, sample purification, sample amplification, sample hybridization, and sample detection could be included in these reagent cartridges 180 or 200. For example, a well 190 or 210 could contain the actual sample, buffer or buffers, oligos, enzymes, or waste. The wells could also contain reagents to help with the detection of nucleic acid binding. One or more of the wells could be reserved (preferably empty) for collection of examined sample or for used reagents. The wells can be sealable and resealable, not only with a foil or tape like substances, but with more traditional lids as well. In one embodiment, every other well is empty, to receive spent reagent from the previous step. In another embodiment, there are at least two empty wells and preferably at least one or two wash solutions in other wells.

In one embodiment, the bottoms of the wells taper down to a point so as to promote the removal of all of the sample from the wells 190 or 210. One advantage of this embodiment is that a predetermined precise amount of sample can be added to each well, thus a very precise and accurate amount of sample or reagent can be added to the electrode array by using the FCC to take all of the sample in the well into the chamber 160. This ability to rapidly add precise amounts of small volumes of liquid to a reaction chamber, especially in an automated system, is one advantage of some of these embodiments. Furthermore, it can be particularly useful in some kits.

The compartments or wells 190 and 210 in the cartridges 180 and 200 can be arranged in various manners. For example, they can be arranged into linear array or rotational arrays as shown in FIG. 4A and FIG. 4B. The linear array can be a commercially available 8-well strip from a microtiter plate. Any arrangement can be used, as long as it allows access, by the flow cartridge tip, to the sample in the compartments.

In some embodiments, the reagent cartridges are associated with fluid mixing or agitating devices. For example, a gas can be bubbled up through each of the wells in the cartridge, or the entire cartridge can be sonicated to promote mixing of the solutions. These additions can be advantageous in situations where the FCC does not have an internal chamber, for example, when the electrode array is attached to an external surface of the housing. This added energy to the solution in the reagent cartridges can promote the removal or mixture of any solution on the FCC.

### General FCC and RC Electrochemical Array Use

In some embodiments, the protocols for using the FCC 100 involve three steps. First, the reagent is aspirated into the FCC, second, the reagent is incubated in the FCC at a certain temperature for an amount of time, and third, the reagent is dumped into a waste container. As will be appreciated by one of skill in the art, not all of the steps have to be performed in each use of the FCC. For example, a particular temperature need not be used or altered; alternatively, the entire last step can be left out if not required. Additionally, as will be appreciated by one of skill in the art, additional steps can be added, including wash steps, and the basic cycle of steps repeated multiple times with either the same or different protocols or reagents for each step.

In some embodiments, a diagnostic assay can be performed with these FCCs 100. For example, in one embodiment, the FCC 100 moves over a reagent cartridge 180, punches through a seal, such as a sealing tape, and makes a fluidic connection to the reagent in a well 190 through a pipette tip or needle. Then, by pulling or pushing a plunger in a pump connected to the second opening 140, fluid can be pumped in or out of the FCC 100.

In some embodiments, each time one desires to add or remove a sample or reagent from the FCC the solutions are changed by moving the FCC 100 to a new well 190 or sample location. Thus, the entire housing 110 and electrode array 170 are moved to the new sample or reagent locations, rather than bringing the new sample or reagent to the device 10 and then across the electrode array. In some embodiments, all that is needed to collect the sample is to place the first opening 130 of the FCC 100 into a solution in a well 190 of a RC 180. Then, a negative pressure can be applied to the second opening 140 to collect the solution into the FCC 100. The amount, both duration and magnitude, of the pressure (including the volume of gas moved), can be altered to control the amount of sample collected. This amount can be either observed, inferred from forces applied, or estimated by the particular forces to be applied. Thus, the amount of each sample in the flow cell cartridge 100 can be known or predicted. Alternatively, the collection of a sample of a known volume in a known amount will also allow one to determine the amount of sample in the flow cell 100. If using a syringe pump, the volume of the syringe can be matched to the volume of the FCC 100 to precisely and repeatably move a predetermined volume of liquid into and out of the FCC 100.

As will appreciated by one of skill in the art, the volume collected in the FCC 100 is not set to any particular minimum for sample collection or removal. In one embodiment the movement of less than 0.1 microliter into or out of the FCC 100 is accurately and precisely collected or eliminated from the FCC by the manipulation of pressure to the second opening 140. The accurate or precise movement of a solution can be of any amount; for example, 0.001-0.01, 0.01-0.1, 0.1-0.2, 0.2-0.4, 0.4-0.6, 0.6-0.8, 0.8-1, 1-2, 2-4, 4-6, 6-8, 8-10, 10-20, 20-40, 40-60, 60-80, 80-100, 100-1000, or 1000-10,000 microliters can be manipulated, as well as larger or smaller volumes. As will be appreciated by one of skill in the art, the limitations on control of volume flow, for both accuracy and precision, will largely depend upon the control that one has on the pressure exerted on the second opening 140, and the size of the first opening 130. As the source of pressure can be changed at the second opening, this variable can be adjusted as needed. In one embodiment, the second opening 140 is connected to two separate sources of vacuum, one for large volumes and one for smaller volumes. As discussed elsewhere, the first opening 130 can actually have a pipette tip attached to it; thus, by altering the pipette tip, one can control the size of this opening.

As will be appreciated by one of skill in the art, while the detection of target sequence or other analyte is occurring, there should be enough sample or solution across the electrode array 170 so that a circuit is completed between the reference electrode 172 and the working electrode 171. Additionally, if each working electrode 171 detects a different target sequence, and each sequence is being assayed, each of these electrodes should contact the solution. However, one-hundred percent of each of the electrodes 171 and 172 need not be covered for all assay formats. Additionally, while reactions are occurring and samples or reagents being added to the FCC 100, the solution or samples need not enter the chamber 160, for example, the solutions can be collected primarily in the first passageway 120. Occasionally, shaking or agitating the entire FCC 100 can be useful to help guarantee that everything is mixed thoroughly.

In one embodiment, air is taken up through the first opening 130, to pull the sample or solution out of the first opening 130 and further into the FCC 100. This allows tips attached to the end of the FCC to be changed. It also allows a greater degree of secureness of the sample. This can also allow for an extremely rapid mixing of samples or reagents together, if this is done multiple times. For example, if a first sample is in the passageway 120 and is pulled up higher into the passageway by applying force to the second opening 140, then a second sample can be collected in the passageway 120 as well. The two samples will not mix as there will be a gap between the two solutions. Then, by pulling both samples up into the chamber 160, where the gap is removed, they will mix in the presence of the electrode array 170, allowing mixture and measurement to occur at similar points in time.

In some embodiments, the FCC is a single use device that is disposable.

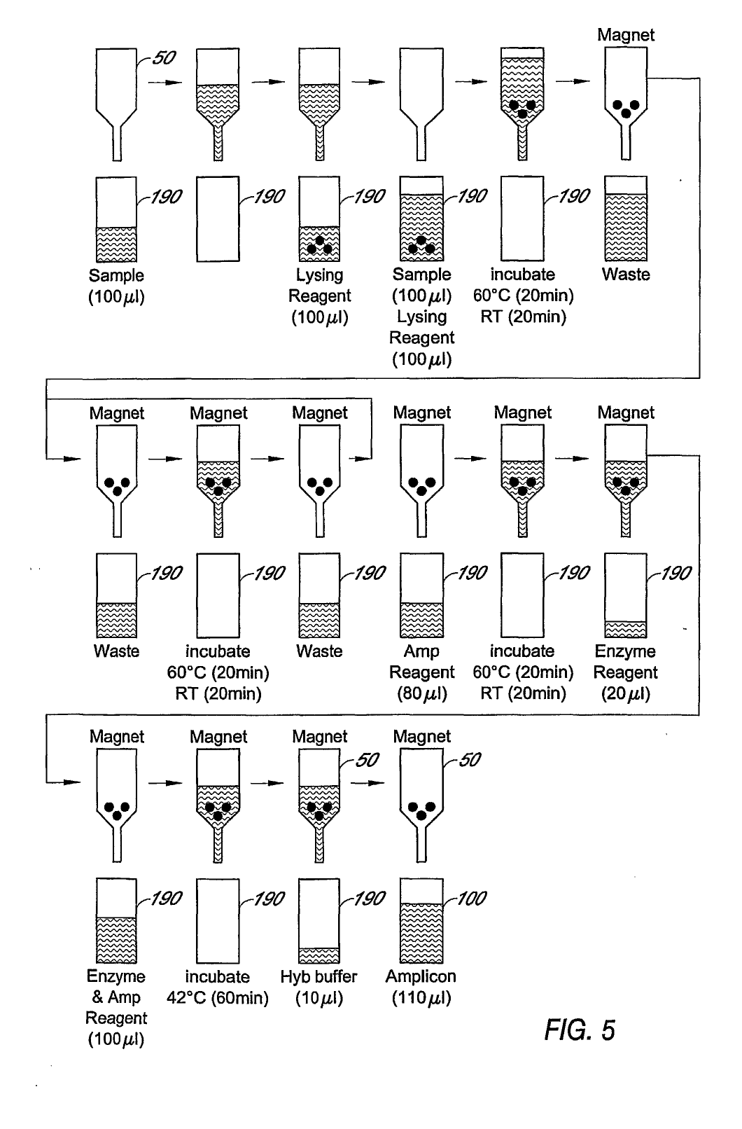

In some embodiments, with reference to FIGS. 4A and 5, an assay starts with a sample containing various nucleic acids, including a target nucleic acid, being collected by the FCC 100 from a first well 190 of an RC 180. Within the FCC 100, the sample can be mixed with purification beads. The beads can be coated with nucleic acids that are complementary to the target nucleic acids, thus purifying the target nucleic acid. As the beads are meant to purify the sample, the purification sequences on the beads could be very general, perhaps to nucleic acids in general or more particular, for example, to a common aspect of the particular nucleic acids. In one embodiment, a very general means for collecting nucleic acids is used on the beads; such a general means can allow the maximum amount of target sample to be retained, which can later be detected on the electrodes. In another example, the beads can isolate only a nucleic acid sequence of a gene of choice. This can reduce any secondary reactions that can occur and can increase the ability of the system to detect very small variations in the genetic material. Finally, the beads could be subtractive, removing interfering or unwanted sequences, enzymes, or other materials.

In some embodiments, the solution that is taken into the chamber can have conditions such that it promotes the binding of the target sequence to the beads. After the target sequence, e.g., DNA or mRNA, in the sample is given an opportunity to bind to the beads, the solution can be expelled from the chamber into a waste well, or second well, 190 of the RC 180. Following this, the chamber 160 could be washed with a reagent taken from a wash well, or third well 190, in the RC 180. Following this, a second solution, perhaps an elution solution, can be taken from a fourth well 190 in the RC 180 to remove the nucleic acids from the beads. Following this, the beads can be removed, such as into the second well or a different waste well 190, so that they do not interfere with the binding of the target sequence to the electrodes 170. Additionally, as this last solution can inhibit binding of the sample to the electrodes, additional salts or reagents can be collected from a fifth well 190 and added to the sample to again allow hybridization to occur, once the beads have been removed. Alternatively, the sample can be expelled into a collection well 190 and adjusted externally to the FCC 100.

In one embodiment of the present invention, the wells 190 can contain both liquid and non-liquid materials. Thus, for example, a first liquid such as sample could be present in a first well. The sample could be drawn into the chamber 160 and transferred to a second well, which can contain a liquid or dried reagent. The sample mixed with the reagent could then incubate in the second chamber before being moved back into the chamber 160. A large number of permutations of sample or reagent movement, incubation, solubilization, mixing, reaction, binding, purification, and the like can be simply and quickly performed by moving liquids into and out of the chamber 160 and into and out of the wells 190, while moving the first opening 130 and/or the wells 190 relative to each other. Mixing and solubilization can be enhanced, for example, by repetitively moving liquid into and out of the chamber 160 and/or a well 190. The steps of the assay and the fluid movement can be controlled and coordinated manually or, preferably, through use of simple electromechanical equipment (e.g., syringe pump and turntable) under the control of a conventional processor such as a computer or a microcontroller, all of which can be considered a part of certain embodiments of the present invention.

In some embodiments, PCR ingredients can be added to the sample solution, such as primers, enzymes, and salt adjustments, so that the target sequence can be amplified. These ingredients can all be stored in a single well, 190, or in multiple wells.

The volume collected can be 100% of the volume in the wells, or it can be controlled by the amount of pressure applied to the second opening 140. Thus, substantially accurate or precise amount of any sample can be added to the chamber through either the modulation of pressure at the second end 140, e.g., as through a pipette, or through the precise or accurate placement of a sample or reagent into the wells 190 and the complete removal of the sample from the wells. Thus, PCRs, rolling circle amplifications or any reaction in general can be performed in the FCC. Following the PCR reaction, the proper solution requirements can be again be obtained by collecting various salts or buffers from particular wells and adding them to the chamber 160 by the application of a negative pressure to the second opening 140. These solution adjustments can be required so that the target sequences can anneal to the PNA or DNA on the electrodes.

In some embodiments, once the target sequence is annealed to the electrode, additional substances, such as ruthenium complexes, can be added to the solution in order to aid in the detection. The ruthenium complexes can be collected from a well 190 by again dipping the first opening 130 into a solution of the complexes and applying a negative pressure to the second opening for a period of time sufficient to draw the desired volume of solution into the FCC 100. Of course, these complexes can be added before the target sequence actually anneals to the electrodes.

In some embodiments, while the electrical potential or current at the electrodes is being monitored, the temperature or other conditions that influence hybridization can be altered to alter the hybridization characteristics of the target sequence. The stronger the hybridization (which will be a function of sequence similarity between the target sequence and the probe sequence on the electrode) the longer the target sequence will be able to bind to the sequence on the probe under increasingly stringent hybridization conditions. Thus, sequence identity will be a function of hybridization, which will be monitored through charge association around the electrodes. In one embodiment, the hybridization potential of the solution is altered by collecting reagents, e.g., salts, from a well 190 periodically throughout the measurements. As the amount collected and the amount in the FCC 100 are both known, the final condition, e.g., ionic strength, of the sample solution will also be known. As the volume added can be small, it can be desirous to agitate the FCC 100 or the sample inside to encourage the solutions to mix.

In certain embodiments, it is advantageous to keep the flow cell cartridge stationary and move the reagent cartridge to the flow cell cartridge. Particularly, the movement could be simpler if the reagent compartments are arranged in a rotational array, as shown in FIG. 4B.

In some embodiments, the FCC 100 and RC 180 combination is not assay specific. The combination can perform basic steps such as sample loading, washing, incubating, mixing, and etc. To switch between different assays performed on the cartridge, one can simply change the reagents in the reagent cartridge and the sequence of how the flow cell interacts with the reagent cartridge. These embodiments can allow for universal cartridges for different diagnostic assays

In some embodiments, the FCC is configured for use on handheld systems. The simplicity of the fluidic handling with this approach and the electrochemical readout provide an opportunity to develop a handheld system for point-of-care or home testing.

### Nucleic Acid Detection Involving the Use of the FCC

Some embodiments include a method of detection of polynucleotide hybridization via an electrochemical array using the FCC 100. Preferably, such an electrochemical array detects nucleic acid hybridization using the general technique of Steele et al. (1998, Anal. Chem. 70:4670-4677).

Typically, in carrying out this technique, a plurality of nucleic acid probes which are complementary to a sequence of interest are used and are attached to the electrodes 171, as shown in FIG. 3A. In certain preferred embodiments, probes range in length from about 10 to 25 base pairs, with a length of about 17 base pairs being most preferred. In some embodiments, the probe strands are positioned within a detection zone. In some embodiments, the detection zone includes a surface, such as on an electrode 171, in contact with a liquid medium, wherein the probe strands are immobilized on the surface such they are also in contact with the liquid medium.

In further carrying out this technique, a target strand (a nucleic acid sample to be interrogated relative to the probe) can be contacted with the probe in any suitable manner known to those skilled in the art. For example, a plurality of target strands can be introduced to the liquid medium described above and allowed to intermingle with the immobilized probes on the electrodes. Preferably, the number of target strands exceeds the number of probe strands in order to maximize the opportunity of each probe strand to interact with target strands and participate in hybridization. If a target strand is complementary to a probe strand, hybridization can take place. Techniques for adjusting the stringency of hybridization and techniques for detecting hybridization are also discussed herein.

Further, embodiments can include any combination of the following steps: extracting a biological sample from a patient or sample, purifying a nucleic acid from a biological sample, amplifying a nucleic acid, isolating a nucleic acid in single stranded form, cyclizing a nucleic acid, elongating a nucleic acid, controlling hybridization stringency, amplifying the nucleic acid on a chip, and detecting hybridization. Each of these steps can occur within the flow cell cartridge described above. Accordingly, such embodiments for each of these steps are discussed in the following sections.

References to extracting an oligonucleotide from a patient typically refers to obtaining a sequence that will form the basis of a target strand. However, in many embodiments, the same techniques, or those which are similar, will also be appropriate for obtaining a sequence that will form the basis of a probe strand that is to be attached to the electrode 171. Those of skill in the art will recognize that various biological and/or artificial sources of oligonucleotides are available and will be able to decide which are most suitable for creating probes or targets depending on the particular goals of the assay to be conducted.

### Extracting a Biological Sample

A variety of methods for extracting nucleic acid from various biological samples from a patient can be used. Biological samples that can be used include any sample from a patient in which a nucleic acid is present. Such samples can be prepared from any tissue, cell, or body fluid. Examples of biological cell sources include blood cells, colon cells, buccal cells, cervicovaginal cells, epithelial cells from urine, fetal cells or cells present in tissue obtained by biopsy. Exemplary tissues or body fluids include sputum, pancreatic fluid, bile, lymph, plasma, urine, cerebrospinal fluid, seminal fluid, saliva, breast nipple aspirate, pus, amniotic fluid and stool. Useful biological samples can also include isolated nucleic acid from a patient. Nucleic acid can be isolated from any tissue, cell, or body fluid using any of numerous methods that are standard in the art.

In some preferred embodiments, a stool sample is taken from a patient as part of a method of screening for colorectal cancer. In particular, methods of extracting biological samples from stool are described in U.S. Pat. No. 5,741,650 (Lapidus et al.). Lapidus et al. teach sectioning a stool sample to extract cells and cellular debris that can be indicative of cancer or precancer. Such a method can be used to obtain biological material containing a nucleic acid for further use in accordance with some of the present embodiments.

For example, with an appropriate tip attached to the FCC, the sample can be withdrawn directly into the FCC 100. For example, blood can be one such sample which could easily be gathered by this means. Such embodiments allow for the direct collection and testing of a sample without the need for additional vessels.

Purifying Nucleic Acid from a Sample

A variety of techniques can be used to purify nucleic acids. Suitable nucleic acids can include DNA (e.g., genomic and circular) and RNA (e.g., mRNA and miRNA). The particular nucleic acid purification method can typically depend on the source of the patient sample and the type of nucleic acid. Techniques for purifying nucleic acids are known in the art and can include the use of homogenization, centrifugation, extraction with various solvents, chromatography, electrophoresis, and other known techniques.

In some preferred embodiments, the biological sample is a stool sample and nucleic acid from colorectal tissue is isolated and purified from stool cross sections according to methods disclosed in U.S. Pat. No. 6,406,857 (Shuber et al.).

In one embodiment, the interior of the FCC 100 contains a means for purifying a sample. For example, there can be a solvent in the FCC 100 to which the sample is added, which will allow purification of the sample. Alternatively, the FCC 100 can contain additional aspects that allow for purification to occur within the FCC, such as antibodies attached to the sides of the inside of the FTC or to beads that are contained within the FTC. In one embodiment, the FCC 100 contains magnetic beads coated with amino acids or nucleic acids which will bind to a desired target. Thus, a sample can be directed gathered through the use of the FCC, and can be further purified through the use of additional aspects within the FCC.

### Amplification of Nucleic Acid

Various techniques which are known in the art can be used to amplify a nucleic acid when practicing the present invention. RCA is one technique that can be used, although PCR is preferred. In one embodiment, a “digital PCR” technique is used. Digital PCR refers to a PCR method in which a liquid sample containing nucleic acids of interest is so thoroughly diluted and partitioned that each partition contains at most one nucleic acid molecule. Accordingly, if subsequent PCR amplification on a partition is successful, all of the resulting strands will be derived from one strand. Hence all of the PCR products for a given partition will be identical. Because the partitions themselves are unlikely to be identical to all the other partitions, it will often be advantageous to study those partitions found to contain nucleic acids in separate assays to determine which warrant further attention.

Digital PCR is discussed in greater detail in Vogelstein et al. “Digital PCR,” Proc. Natl. Acad. Sci. USA, Vol. 96, pp. 9236-41, August 1999. In one embodiment, this dilution process is achieved through the use of the FCC 100. For example, the sample can contain multiple RNA sequences, only one of which is the desired target RNA sequence. The entire sample will be taken into the FCC and a volume of solvent will then be added to the FCC as well, thus diluting the sample. Following this, most of the diluted sample will be expelled from the FCC; however, a small volume of the sample will remain in the FCC. To this small volume, an additional volume of solvent will be added, via the described use of the FCC 100, to further dilute the sample concentration. These steps can be repeated until the sample is appropriately diluted.

In some embodiments, the FCC 100 also contains a Peltier device (or other thermal source or sink) by which the sample can be heated and cooled, which, when combined with being able to add additional reagents to the FCC, will allow for PCR to occur within the FCC without the need for a separate device. In these embodiments, the FCC allows for amplification via PCR. Thus, a sample can be gathered, purified and then amplified, all within a single FCC. The temperature can be controlled, either through an external source, or through a Peltier device inside of the FCC 100. Additionally, the temperature of the solution can be monitored through the use of a temperature sensor 155.

### Isolating Single Stranded Nucleic Acid

Various techniques are known in the art for producing or isolating single stranded nucleic acid from samples containing double stranded nucleic acid.

In one method, single stranded nucleic acid is isolated using a streptavidin-coated bead. In performing this technique, an amplification product is denatured to generate single-stranded products, wherein at least one strand contains an addressable ligand at one terminus. In some preferred embodiments, a biotinylated single-stranded PCR product having a copy of the nucleotide sequence of interest is incubated with streptavidin-coated beads, under conditions such that the biotinylated PCR product is attached to a bead, forming a bead-target sequence complex.

In other preferred embodiments, one strand of a double stranded nucleic acid is removed, for example, by selective exonuclease digestion. The remaining single stranded nucleic acid can further be used in accordance with the present invention.

For these procedures and methods, the FCC can contain the coated beads or readily accept the isolated product from the beads, which can be located, for example, in a well of the RC 190. Thus, in some embodiments, the sample can again be purified by performing a PCR reaction within the FCC 100 and then selecting those PCR products through the use of streptavidin-coated beads in the FCC or through the use of the FCC.

### Cyclizing the Nucleic Acid and Performing RCA

In some embodiments, in performing an assay, one cyclizes and elongates the target nucleic acid prior to hybridization. “Cyclization” generally refers to the process of creating a polynucleotide circle (preferably containing a particular sequence), while “elongation” generally refers to the process of increasing the length of a polynucleotide. In preferred embodiments, elongation includes a rolling circle amplification (RCA) step with an appropriate polynucleotide circle and is used to create a long strand of target nucleic acid.

In particular, cyclization and elongation can be used to generate one or more long target strands in which a sequence being interrogated is repeated several times. Effectively, many copies of a small target strand are linked end to end to generate a large target strand. Although cyclization/elongation can be used to add as little as one repetition, it is generally preferred that multiple repetitions be added, for example, approximately 10, 50, 100, 250, 500, 750, or 1000 repetitions or more can be attached. Circle size is also adjustable according to the requirements of the assay. Preferred circle sizes are in the range of about 40 to about 1000 base pairs, with about 800 base pairs being most preferred. Notably, the number of repetitions selected can depend on the length of the circle being used. Specifically, it will generally be preferable to use more repetitions with smaller circles and fewer repetitions with larger circles so that the strands produced will be appropriately manageable and functional according to the demands of the assay.

Generally, any one of the many repetitions of the sequence on a large strand would be able to hybridize to a probe just as if that sequence were alone on a standard short target strand. Further, just one large target strand can generally hybridize to multiple probes (by coiling back toward the electrode surface and allowing another identical region of the long strand to attach to another complementary probe).

In some embodiments, elongation and the use of long target strands have various advantages. Particularly favorable advantages are related to stringency. “Stringency” refers to a measurement of the ease with which various hybridization events can occur. For example, two strands that are perfectly complementary generally form a more stable hybrid than two strands that are not. Various stringency factors (such as temperature, pH, or the presence of a species able to denature various hybridized strands) can be adjusted such that in a single environment, the perfectly complementary pair would stay together while the imperfect pair would fall apart. Ideal conditions are generally those which strike a balance between minimizing the number of hybridizations between noncomplementary strands (false positives) and minimizing the number of probes which remain unhybridized despite the presence of eligible complementary target strands (false negatives). Other various techniques for controlling stringency are also discussed in the next section.

Elongation is one technique that is useful in improving the effectiveness of temperature as a stringency factor. A perfect hybrid is typically more stable than an imperfect hybrid and will outlast the imperfect hybrid when the temperature is increased. However, dehybridization in either case is not a single event when dealing with populations of molecules. Instead, more and more molecules give up the hydrogen bonds that hold opposing base pairs together over a range of temperature. Perfect hybrids outlast imperfect hybrids, but it is often very difficult, if not impossible, to find a single temperature at which there are no imperfect hybrids while perfect hybrids abound.

Longer nucleic acid molecules exhibit a less gradual transition between their hybridized and unhybridized states when the temperature is changed. This is to say that the melt curve for a given population of molecules is steeper and more decisive when the nucleic acid strand is longer. However, the distance between the curves of perfect and imperfect hybrids of equivalent length tend to crowd in a smaller temperature range, frustrating the initial attempt to create a stringency environment that will distinguish between them.

The use of elongated target strands that can hybridize to multiple probes allows a larger stringency range. In other words, the melt curves are steeper (than those of the short molecules) and that the distance between the melting temperatures of perfect and imperfect strands are farther apart (than those of the long molecules). In these situations, the melt curves are steep and the Δ Tm is large. This combination facilitates improved specificity in the assay because of the large temperature range in which matched duplexes generally exist and mismatched duplexes do not.

Accordingly, some embodiments include cyclization and elongation steps to produce a target strand of increased length. Preferably, a sequence is repeated several times on the target strand such that one target strand can participate in hybridization with more than one immobilized probe. As this can be used to create a larger temperature window in which perfect hybrids remain and imperfect hybrids fall apart, it is advantageous to adjust the temperature of the assay environment to minimize false positives as well as false negatives.

In some embodiments, it is advantageous to perform an assay in which hybridization is evaluated at two or more different temperatures. For example, where a duplex polynucleotide with a single base mismatch has a melting temperature Tₘ₁ and a duplex polynucleotide with no base mismatch has a higher melting temperature Tₘ₂, it is possible to first detect whether the duplex exists at a temperature below Tₘ₁, then increase the temperature of the assay environment above Tₘ₁ to detect whether the duplex exists at a temperature between Tₘ₁ and Tₘ₂. In this case, the results of such an assay could indicate whether a single base mismatch exists in the duplex being interrogated. In some cases, a determination of the temperature at which a duplex falls apart can be used to evaluate the quantity, type, and/or location of mismatches, if any. Various techniques for detecting hybridization are discussed infra.

In some embodiments, the FCC 100 is used to detect and monitor the above described form of hybridization. One can first allow hybridization of the target sequence to the probes on the electrodes at a lower temperature. The temperature of the solution is controlled by a Peltier device. After hybridization, the temperature of the solution is increased until the target sequence falls off of the electrode. The temperature of the solution at the point in time where target sequence falls off of the probe is observed through the temperature sensor 155. The temperature at which the sequences fall apart, or off of the probe, is used to determine the number, type, and/or location of any mismatches. In one embodiment, the temperature is compared to a control or standard target sequence with a known number, type, and/or location of mismatches.

Those of skill in the art will appreciate that other techniques to elongate nucleic acids, including for example, head-to-tail polymerization, can also be used to achieve favorable results with regard to temperature stringency.

In some embodiments, it is advantageous to use “padlock probe” and/or “addressed amplicon” techniques when generating a target strand that can be hybridized to a probe strand. These techniques are discussed in greater detail in U.S. Pat. Pub. No. 20040086892 entitled “UNIVERSAL TAG ASSAY,” filed Apr. 24, 2003. Some embodiments of the present invention include providing a polynucleotide sample and then performing an assay to determine whether it contains a sequence of interest. In some such assays, a nucleic acid circle is prepared in connection with the polynucleotide sample that contains both a portion of a sequence complementary to the sequence being interrogated and an “address sequence.” The address sequence is typically an arbitrary sequence of nucleotides that will also appear on a probe strand. The circle can be amplified by RCA to produce a long target that contains several repetitions of the complement to the address sequence. When the target strand is allowed to interact with a probe containing the address sequence, the two can hybridize. Detection of hybridization can be used as an indication of the presence of the sequence being interrogated in the original sample. It will be appreciated that assays of this type can detect the presence of various sequences as well as the presence of single nucleotide polymorphisms (SNPs). When detecting SNPs, for example, it can be advantageous to use different cyclizable strands containing each of the possible nucleotides at the suspected SNP location. Each cyclizable strand should also have a unique address sequence. The one cyclizable strand that fits correctly with the sample can then cyclize and undergo amplification. Then, by determining which address sequence corresponds to a probe-target hybrid, the identity of the nucleotide at the SNP location can be determined.

As embodiments of the FCC 100 allow for the rapid exchange and mixing of samples, and in some embodiments temperature control and purification abilities, some embodiments of the FCC are readily able to achieve the above reactions, pH changes, and temperature changes in order to facilitate the above method.

### Controlling Hybridization Stringency

In one embodiment, in performing a hybridization step, it is preferable to introduce single stranded targets derived as described above to the liquid medium such that they can hybridize with probes immobilized on an electrode. Preferably, the number of target strands used in an assay will exceed the number of probe strands in order to maximize the opportunity of each probe strand to interact with target strands and participate in hybridization. If a target strand is complementary to a probe strand, hybridization can take place when the two come into contact. However, in some cases, even strands which are not truly complementary can come together and stay together as an imperfect hybrid. Whether or not various hybridization events occur can be influenced by various stringency factors such as temperature, pH, or the presence of a species able to denature various hybridized strands. Increasing the quantity of target strands is one technique that can be useful in minimizing the number of probes that should hybridize to targets, but do not (false negatives).

Preferred techniques for controlling stringency include setting and maintaining the temperature and pH of the liquid medium environment. More preferred techniques also incorporate introducing one or more chemical species as stringency agents that can minimize the number of false positives and/or false negatives. Agents that can be used for this purpose include quaternary ammonium compounds such as tetramethylammonium chloride (TMAC). The use of a Peltier device, for example, is one way that the temperature of the FCC can be controlled and in turn controls the stringency of the hybridization.

TMAC is particularly useful in minimizing false positives. This species generally acts through a non-specific salt effect to reduce hydrogen-bonding energies between G-C base pairs. At the same time, it binds specifically to A-T pairs and increases the thermal stability of these bonds. These opposing influences have the effect of reducing the difference in bonding energy between the triple-hydrogen bonded G-C based pair and the double-bonded A-T pair. One consequence is that the melting temperature of nucleic acid hybrids formed in the presence of TMAC is solely a function of the length of the hybrid. A second consequence is an increase in the slope of the melting curve for each probe. Together, these effects allow the stringency of hybridization to be increased to the point that single-base differences can be resolved, and non-specific hybridization minimized. Various techniques for using a stringency agent such as TMAC are discussed in U.S. Pat. No. 5,849,483 (Shuber).

Further, specific control of stringency factors can be useful in assays which seek to identify mutations occurring at the end of an oligonucleotide fragment. For example, the mutator cluster region of the APC gene, wherein mutations are highly correlated with colon cancer, is approximately 800 base pairs in length. If a probe oligomer is approximately 17 base pairs in length, it will typically require approximately 44 oligomers to blanket the entire 800 base pair strand. Mutations at the end of a fragment are often difficult to detect, so it can be beneficial to use a second series of oligonucleotides that also blanket that 800 base pair strand, but are offset such that the middle of the second series of oligonucleotides corresponds to the ends of the adjacent first series of oligonucleotides. Allowing for a gap of three base pairs between adjacent probe sequences, it will typically require 80 oligomers to test 800 base pairs for mutations. Various high volume techniques for testing a mutator cluster region can be used. In a preferred embodiment, standard multiwell plates having 96 wells and 20 electrodes per well can be used to test a particular region; assuming four wells are used to determine which one of the four bases appears at a particular point in the sequence, each 96 well plate can test the properties of 24 different molecules.

Further, temperature dependence can be adjusted by varying the length of individual oligonucleotides since longer sequences tend to be more stable. Oligonucleotides that are to be used in an assay need not all be the same length.

As described above, some of the embodiments of the FCC are readily capable of adjusting the pH and temperature of the solution in the chamber; thus, the FCC is an ideal device by which to achieve the above steps. Any of the above stringency agents can be added to the hybridized target sequences by placing the first opening 130 into a solution of the stringency agent and applying an appropriate amount of negative pressure to the second opening 140.

### Amplifying the Hybridized Nucleic Acid—On-Chip Amplification

In some embodiments it can be advantageous to augment the signal created by the target strand that indicates hybridization has occurred. One method for doing this is to elongate the target strand after it has hybridized to the probe. This technique can be referred to as “on-chip” amplification. Two methods for on-chip amplification are particularly preferred. Both of which are easily performed with the use of some of the embodiments of the FCC 100.

In one method, either the 3′ or 5′ end of the hybridized PCR product can be targeted for a head-to-tail polymerization that builds up the amount of DNA on the electrodes. Typically, three different oligonucleotides (not counting the immobilized probe and the target strands) will be used as shown here: the first oligomer is complementary to the 3′ end of the hybridized PCR product (targeting the complement of the primer sequence), and contains a sequence A at its 5′ end; the second oligomer has a sequence 5′-A*B-3′, where A* is complementary to A; the third oligonucleotide has sequence 5′-AB*-3′. These oligomers can form a polymeric product. The head-to-tail polymerization can continue until the strand reaches a desired length. Generally, when performing head-to-tail polymerization, the ultimate length of the polynucleotide is limited in part by a competing cyclization reaction of the head-to-tail oligomers. A higher concentration of head-to-tail oligomers in the liquid medium will generally produce longer linear polymers attached to the electrode, however.

Another method of on-chip amplification method uses rolling circle amplification. Preferably, a preformed circle (approximately 40 to 300 nucleotides) that has a region complementary to the 3′ end of the bound PCR product is hybridized to the PCR product as shown. A processive DNA polymerase can then be added so that RCA results, elongating the bound PCR product. Preferably, the PCR product is elongated by approximately 10 to 100 copies of the circle.

Another technique for on-chip amplification can be used in conjunction with other on-chip amplification methods and is commonly referred to as “branch” amplification. Here, additional polynucleotides that are capable of hybridizing with the target strand in a region beyond the probe-target hybridization region can be added to the liquid medium and allowed to hybridize with the bound target to further increase the amount of bound polynucleotide material when probe-target hybridization occurs. Preferably, these branch polynucleotide strands are further amplified. Further, when a branch amplification technique is used, it can be advantageous to attach branches on top of branches, a technique known as hyperbranching. Additional discussion of branching and hyperbranching techniques can be found, for example, in: Urdea, Biotechnology 12:926 (1994); Horn et al., Nucleic Acids Res. 25(23):4835-4841 (1997); Lizardi et al., Nature Genetics 19, 225-232 (1998); Kingsmore et al. (U.S. Pat. No. 6,291,187); Lizardi et al. (PCT application WO 97/19193).

After performing an on-chip amplification, the increased amount of DNA can generate a larger and more detectable signal. This can be advantageous for assay purposes since both the probe and the target typically produce some detectable signal. If the signal of the target is enhanced, the contrast between hybridized and unhybridized probes will be more profound. In some embodiments, however, nucleic acid analogs can be used as probes which do not contribute to the overall signal; such designs are discussed in the following section. Even when such nucleic acid analogs are used as probes, target elongation can still be desirable.

Preferably, nucleic acid hybridization is tested electrochemically using a transition metal complex. More preferably, hybridization is detected by measuring the reduction of a ruthenium complex as described below.

### Detecting Hybridization

Various techniques can be used to determine whether hybridization has occurred in a FCC 100. As indicated above, preferred embodiments of the present invention feature the use of a transition metal complex. In particular, a ruthenium complex can be used as a counterion to conduct an electrochemical assay using the general technique of Steele et al. (1998, Anal. Chem. 70:4670-4677).

Counterions, such as Ru(NH₃)₆³⁺ or Ru(NH₃)₅py³⁺, can be introduced to the liquid medium surrounding the immobilized oligonucleotides. Typically, Ru(NH₃)₅py³⁺ is preferred because its reduction to a divalent ion does not occur at the same electrical potential as the reduction of molecular oxygen. These compositions can be added to the sample in a FCC in a manner described above; thus, the counterions need not be present except for during the actual detection step itself.

Once introduced, the counterions will tend to cloud around the negatively charged backbones of the various nucleic acid strands. Generally, the counterions will accumulate electrostatically around the phosphate groups of the nucleic acids whether they are single or double stranded. However, because a probe and target together physically constitute a larger amount of nucleic acid than the probe alone, the hybridized nucleic acid will typically have more counterions surrounding it. In general, the target can be much longer than the probe, typically 2 to 100 times, in which case the counterion accumulation will be dominated by single stranded regions of the target.

In one embodiment, the signal contrast between single stranded and double stranded nucleic acid is increased by limiting the electrical signal from the probe strands. In particular, this can be done by limiting the electrical attraction between the probe strand and the counterions which participate in electron transfer. For example, if the probe strands are constructed such that they do not contain a negatively charged backbone, then they will not attract counterions. Accordingly, more of the detectable signal will be due to counterions associated with the target strands. In cases where hybridization has not occurred, the detectable signal will be measurably lower since the target strands are not present to participate in counterion attraction.

Probe strands without a negatively charged backbone can include peptide nucleic acids (PNAs), phosphotriesters, and methylphosphonates. These nucleic acid analogs are known in the art. Thus, in one embodiment, the probe strands attached to the electrodes 171 in the electrode array 170, in the chamber 160 of the FCC 100 are PNA probe strands.

In particular, PNAs are discussed in: Nielsen, “DNA analogues with nonphosphodiester backbones,” Annu Rev Biophys Biomol Struct, 1995; 24:167-83; Nielsen et al., “An introduction to peptide nucleic acid,” Curr Issues Mol Biol, 1999; 1(1-2):89-104; Ray et al., “Peptide nucleic acid (PNA): its medical and biotechnical applications and promise for the future,” FASEB J., 2000 June; 14(9):1041-60.

Phophotriesters are discussed in: Sung et al., “Synthesis of the human insulin gene. Part II. Further improvements in the modified phosphotriester method and the synthesis of seventeen deoxyribooligonucleotide fragments constituting human insulin chains B and mini-cDNA,” Nucleic Acids Res, 1979 Dec. 20; 7(8):2199-212; van Boom et al., “Synthesis of oligonucleotides with sequences identical with or analogous to the 3′-end of 16S ribosomal RNA of Escherichia coli: preparation of m-6-2-A-C-C-U-C-C and A-C-C-U-C-m-4-2C via phosphotriester intermediates,” Nucleic Acids Res, 1977 March; 4(3):747-59; Marcus-Sekura et al., “Comparative inhibition of chloramphenicol acetyltransferase gene expression by antisense oligonucleotide analogues having alkyl phosphotriester, methylphosphonate and phosphorothioate linkages,” Nucleic Acids Res, 1987 Jul. 24; 15(14):5749-63.

Methylphosphonates are discussed in: U.S. Pat. No. 4,469,863 (Ts'o et al.); Lin et al., “Use of EDTA derivatization to characterize interactions between oligodeoxyribonucleoside methylphosphonates and nucleic acids,” Biochemistry, 1989, Feb. 7; 28(3):1054-61; Vyazovkina et al., “Synthesis of specific diastereomers of a DNA methylphosphonate heptamer, d(CpCpApApApCpA), and stability of base pairing with the normal DNA octamer d(TPGPTPTPTPGPGPC),” Nucleic Acids Res, 1994 Jun. 25; 22(12):2404-9; Le Bec et al., “Stereospecific Grignard-Activated Solid Phase Synthesis of DNA Methylphosphonate Dimers,” J Org Chem, 1996 Jan. 26; 61(2):510-513; Vyazovkina et al., “Synthesis of specific diastereomers of a DNA methylphosphonate heptamer, d(CpCpApApApCpA), and stability of base pairing with the normal DNA octamer d(TPGPTPTPTPGPGPC),” Nucleic Acids Res, 1994 Jun. 25; 22(12):2404-9; Kibler-Herzog et al., “Duplex stabilities of phosphorothioate, methylphosphonate, and RNA analogs of two DNA 14-mers,” Nucleic Acids Res, 1991 Jun. 11; 19(11):2979-86; Disney et al., “Targeting a Pneumocystis carinii group I intron with methylphosphonate oligonucleotides: backbone charge is not required for binding or reactivity,” Biochemistry, 2000 Jun. 13; 39(23):6991-7000; Ferguson et al., “Application of free-energy decomposition to determine the relative stability of R and S oligodeoxyribonucleotide methylphosphonates,” Antisense Res Dev, 1991 Fall; 1(3):243-54; Thiviyanathan et al., “Structure of hybrid backbone methylphosphonate DNA heteroduplexes: effect of R and S stereochemistry,” Biochemistry, 2002 Jan. 22; 41(3):827-38; Reynolds et al., “Synthesis and thermodynamics of oligonucleotides containing chirally pure R(P) methylphosphonate linkages,” Nucleic Acids Res, 1996 Nov. 15; 24(22):4584-91; Hardwidge et al., “Charge neutralization and DNA bending by the Escherichia coli catabolite activator protein,” Nucleic Acids Res, 2002 May 1; 30(9):1879-85; Okonogi et al., “Phosphate backbone neutralization increases duplex DNA flexibility: A model for protein binding,” PNAS U.S.A., 2002 Apr. 2; 99(7):4156-60.

In general, an appropriate nucleic acid analog probe will not contribute, or will contribute less substantially, to the attraction of counterions compared to a probe made of natural DNA. Meanwhile, the target strand will ordinarily feature a natural phosphate backbone having negatively charged groups which attract positive ions and make the strand detectable.

Alternatively, a probe can be constructed that contains both charged nucleic acids and uncharged nucleic acid analogs. Similarly, pure DNA probes can be used alongside probes containing uncharged analogs in an assay. However, precision in distinguishing between single stranded and double stranded will generally increase according to the electrical charge contrast between the probe and the target strands. Hence, the exclusive use of probes made entirely of an uncharged DNA analog will generally allow the greatest signal contrast between hybridized and non-hybridized molecules on the chip. In general, probe strands containing methylphosphonates are preferred when nucleic acid analogs are desired.

Ru(NH₃)₅py³⁺ is a preferred counterion, though any other suitable transition metal complexes that bind nucleic acid electrostatically and whose reduction or oxidation is electrochemically detectable in an appropriate voltage regime can be used.

Various techniques for measuring the amount of counterions can be used. In some preferred embodiments, amperometry is used to detect an electrochemical reaction at the electrode. Generally, an electrical potential will be applied to the electrode. As the counterions undergo an electrochemical reaction, for example, the reduction of a trivalent ion to divalent at the electrode surface, a measurable current is generated. The amount of current corresponds to the amount of counterions present which in turn corresponds to the amount of negatively-charged phosphate groups on nucleic acids. Accordingly, measuring the current allows a quantitation of phosphate groups and can allow the operator to distinguish hybridized nucleic acid from unhybridized nucleic acid and determine whether the target being interrogated is complementary to the probe (and contains the sequence of interest).

Some embodiments of the present invention allow detection of nucleic acid mutations with improved accuracy and precision. In some embodiments, for example, a mutation can be detected at a level of about 1 part in 10² (which means one mutant version of a gene in a sample per 100 total versions of the gene in the sample) or less, about 1 part in 10³ or less, about 1 part in 10⁴ or less, about 1 part in 10⁵ or less, or about 1 part in 10⁶ or less.

Although electrochemical measurement is a preferred technique for hybridization detection, those of skill in the art will appreciate that many other techniques can also be appropriate. For example, a detectable label can be attached to or otherwise associated with certain polynucleotides in the detection zone. Accordingly, such a label can then be detected as an indication of whether hybridization has occurred. Such labels are well klown in the art and can include, for example, chemical moieties, dyes, radioactive probes, quantum dots, and nanoparticles such as quantum dots. Techniques for detection of various labels can include, for example, chemical detection, radioactivity detection, UV and/or visible spectroscopy, fluorescence, and the like. Again, the use of the FCC 100 could still be beneficial, as, in some embodiments, it will allow the rapid and efficient exchange of samples, reagents, and solutions across the detection zone. Only the detection means of the FCC would be modified. In some embodiments, the detection means is optically based, thus, while the housing 110, would have to be substantially optically transparent for that particular optical signal, the basic arrangement of the FCC 100 (as shown in FIG. 2 or FIG. 3A/3B) could be sufficient. Of course, the electrode arrays need not be electrodes, any substrate to which one could attach probes would be sufficient.

### EXAMPLES

### Example 1

This example demonstrates one method in which one embodiment of a FCC 100 can be used. A FCC 100 is attached to a pipette by the FCC's second opening 140 and a pipette tip is attached to the first opening 130 of the FCC. A first amount of a nucleic acid sample which is to be examined is placed in a solution in a well 190 of a RC 180 and the solution with the nucleic acids is collected through the pipette tip attached to the FCC 100 via suctioning with the pipette. A volume sufficient to allow the sample to contact the electrode array is used. The electrodes 171 in the FCC contain PNA probes that are complementary to the sequence that one desires to detect. Rolling circle amplification is then used to elongate the nucleic acids contained within the target nucleic acids. Reagents for the rolling circle amplification are taken from another well 190 in the RC 180 and into the chamber 160 by placing the first opening 130 into the reagents and applying additional suction to the second end 140 of the FCC 110. The temperature is manipulated through a Peltier device and the temperature monitored through the use of a temperature sensor 155. Following this, the PNA coated electrodes and the target sample are then allowed to hybridize together on the electrodes 171. Excess or undesirable solution is removed by applying a positive pressure at the second opening 140, preferably releasing solution through the first opening 130, into a waste well 190, of a RC 180. A sufficient volume of solution is then added to the chamber 160, so that the electrodes 171 and 172 are electrically connected. Following this, a ruthenium complex is added to the chamber 160 so as to increase the detectable presence of the target sequence, the ruthenium complex is added through the standard use of the FCC 100 and RC 180. Following this, the hybridization stringency of the solution is manipulated by increasing the temperature of the solution through the Peltier device, while monitoring the increase in temperature through the temperature sensor 155. The temperatures at which the target strands fall off of the electrodes, as determined through changes in the potential at the electrodes and the temperature sensor 155, result in a melting curve for the target strand. The melting curve for the target strand is then compared to the melting curves of other known sequences to determine the sequence of the target strand in order to determine the presence or absence of a particular target strand (sequence).

### Example 2

This example demonstrates how a target RNA sequence in a biological sample can be purified, amplified and hybridized using a pipette tip 50 and the FCC 100. FIG. 5 outlines how the FCC is used in this example. The volume (the volume of solution that the entire internal volume of the FCC can contain and control movement thereof) of the FCC is 200 μl.

The 100 μl sample is aspirated into a container, such as a pipette tip 50, and the sample is then dumped into a well 190 with 100 μl lysing reagent and with magnetic beads that can bind to the target RNA sequence. The mixing of the reagents is then enhanced by pushing the mixture in and out of the pipette tip 50 for a few times. The mixture is then aspirated into the pipette tip 50 and incubated at 60° C. for 20 min and then at room temperature for 20 minutes. The reagent is then dumped into a waste well while the magnetic beads and target sample are held in the pipette tip 50 by a magnetic force. The pipette tip 50, the magnets, and the sample attached to the magnet are then repeatedly washed. Following this, amplification reagents (80 microliters) from another well 190 are then added to the pipette tip 50, which still contains the beads and the sample. This mixture is then incubated at 60° C. for 5 minutes and then at 42° C. for 10 minutes allowing primer annealing to occur. Following this, the amplification reagents (80 microliters) are released (without the magnets) into enzyme reagents (20 microliters) well. The amplification reagents and the enzyme reagents are allowed to mix, and are then aspirated into the pipette tip 50 and incubated at 42° C. for 60 minutes. Following this, 10 microliters of a hybridization buffer is then added to the solution, resulting in 110 microliters of amplicon. These 110 microliters of amplicon is then taken into a FCC 100, wherein it is then examined for its sequence characteristics by binding of the target sample to the probe sequences on the electrode 171.

In an alternative embodiment, the entire reaction is carried out in a FCC 100, rather than in both a FCC 100 and a pipette tip 50, as in example 2 above. In such an embodiment, the hybridization buffer is sucked up into the FCC 100, rather than expelling the solution into a well 190 to later be analyzed. In such an embodiment, the protocol would be the same as that depicted in FIG. 5, except that the pipette tip 50 would be replaced with a FCC 100, and the final 110 microliters of solution would be analyzed in the FCC 100 that was used to move samples around in the earlier part of the protocol.

1. A flow cell cartridge, said flow cell cartridge comprising:
a housing forming a chamber, wherein the housing has a first opening adapted for the flow of a liquid and a second opening adapted for the flow of a gas, wherein said openings open to the inside of the chamber and movement of a gas out of said second opening is adapted to move a gas or a liquid into said first opening; and
an electrode array positioned inside said chamber such that a probe on the electrode array is exposed to the inside of the chamber, wherein the electrode array has an electrically conductive connection to a point external to the chamber, and wherein at least a portion of the electrode array has probe nucleic acid bound thereto.

2. The flow cell cartridge of claim 1, wherein the probe nucleic acid comprises peptide nucleic acid segments.

3. The flow cell cartridge of claim 2, wherein the chamber is shaped in two dimensions in a diamond shape.

4. The flow cell cartridge of claim 1, wherein the housing is configured to accept a pipette tip over a portion of the housing section creating the first opening.

5. The flow cell cartridge of claim 4, wherein the chamber has a first opening that is gas permeable and liquid permeable, and has a second opening that is gas permeable but not significantly liquid permeable.

6. The flow cell cartridge of claim 1, wherein the electrically conductive connection to the exterior of the chamber is configured to contact spring loaded pins.

7. The flow cell cartridge of claim 1, wherein the electrically conductive connections to the electrode array comprise pre-deformed metal bumps.

8. The flow cell cartridge of claim 1, further comprising a Peltier device.

9. The flow cell cartridge of claim 1, further comprising a temperature sensor.

10. The flow cell cartridge of claim 9, wherein the temperature sensor is positioned within the chamber.

11. The flow cell cartridge of claim 1, further comprising a resistive heater and a temperature sensor.

12. A flow cell cartridge, said flow cell cartridge comprising:
a housing, said housing comprising 1) a front half and 2) a back half, wherein the front half when joined with the back half forms 3) a chamber, and wherein said housing has a first opening that is both liquid and gas permeable and a second opening that is gas permeable but is not significantly liquid permeable;
at least a working electrode and a reference electrode, wherein said electrodes are in said chamber; and
an electrical connection located on an external surface of the housing, wherein said electrical connection is in electrical contact with one or more of said electrodes.

13. The flow cell cartridge of claim 12, further comprising a Peltier device thermally coupled to said chamber.

14. The flow cell cartridge of claim 13, further comprising a temperature sensor thermally coupled to said chamber.

15. The flow cell cartridge of claim 14, further comprising a nucleic acid probe attached to said working electrode.

16. The flow cell cartridge of claim 15, further comprising a peptide nucleic acid segment attached to said working electrode.

17. The flow cell cartridge of claim 12, further comprising a counter ion.

18. The flow cell cartridge of claim 17, wherein the counter ion is a ruthenium ion.

19. The flow cell cartridge of claim 12, wherein the back half of the housing comprises a substrate for an electrode.

20. The flow cell cartridge of claim 12, wherein the second opening is connected to a vacuum source.

21. The flow cell cartridge of claim 20, wherein the first opening is connected to a pipette tip.

22. A method of applying a sample to an electrode array, the method comprising:
contacting a first opening of a flow cell cartridge (FCC) to a sample to be tested, wherein said FCC comprises a housing, said housing comprising a chamber, said chamber having a first opening and a second opening, and an electrode array contained within said chamber; and
applying a negative pressure to the second opening for a period of time to move the sample through the first opening and into the chamber, thus promoting contact between the sample and the electrode array.

23. The method of claim 22, further comprising incubating a sample or reagent solution in said chamber for a time sufficient for polynucleotide indicative of the presence of analyte in a sample to bind to a probe nucleic acid attached to said electrode, and then expelling said sample or reagent solution from the chamber by exerting a positive pressure through said second opening.

24. The method of claim 22 or 23, further comprising applying a negative pressure to the second opening to bring into the FCC a set of reagents for performing a rolling circle amplification; and performing a rolling circle amplification inside of said chamber.

25. The method of claim 24, further comprising waiting for a period of time sufficient to allow a target nucleotide segment in said chamber that is indicative of the presence of analyte in said sample to bind to a probe nucleic acid attached to said electrode, and determining an electrical signal at said electrode that is indicative of the presence of said target polynucleotide segment.

26. (canceled)

27. A method for performing an assay, comprising:
providing a housing having a first opening, a second opening, a chamber interposed between said first and second opening, wherein said chamber includes at least one binding moiety for indicating a positive assay;
providing at least first, second, and third wells containing liquids or reagents useful in performing an assay;
positioning the first opening in liquid contact with the first well;
moving liquid from the first well through the first opening and into the chamber by removing gas from the second opening;
positioning the first opening in liquid contact with the second well;
moving liquid from the second well through the first opening and into the chamber by removing gas from the second opening;
positioning the first opening in liquid contact with the third well;
moving liquid from the third well through the first opening and into the chamber by removing gas from the second opening; and
ascertaining whether said binding moiety has participated in binding indicative of a positive assay.

28-51. (canceled)
